(19) United States
(12) Patent Application Publication
(10) Pub. No.: US 2026/0199146 A1
(43) Pub. Date: Jul. 16, 2026

(54) DISPOSABLE ORAL SPONGE

(71) Applicant: Katina Renee Cottrell, Englewood, OH (US)

(72) Inventor: Katina Renee Cottrell, Englewood, OH (US)

(21) Appl. No.: 19/017,714

(22) Filed: Jan. 12, 2025

(51) Int. Cl. A61F 13/36; A61F 13/38; A61F 13/84

(52) U.S. Cl. CPC A61F 13/36; A61F 13/38; A61F 13/8405

(57) ABSTRACT

A disposable oral sponge which includes a flexible handle and a sponge head connected to the flexible handle, wherein the flexible handle includes a preloaded amount of fluid for introduction into the sponge head. The flexible handle includes a hollow conduit in communication with a fluid storage area of the handle. A rupturable membrane is disposed in the handle between the fluid storage area and the hollow conduit. The rupturable membrane is rupturable by applying pressure to the membrane and allowing flow of fluid to the the sponge head.

### FIELD OF INVENTION

The present invention relates to the field of oral care. More particularly, the invention relates to providing moisture to a patient’s mouth to assist in health care.

### BACKGROUND OF INVENTION

At present, in connection with treating a patient, such as an elderly or incapacitated or critically ill patient in a hospital or care facility, an oral care implement is provided which generally is a mouth sponge. The mouth sponge includes a sponge connected to an end of a stick. The sponge is typically dipped into a reservoir and inserted into the patient’s mouth to provide added moisture to the mouth without causing aspriation problems. During use, the attendant soaks the sponge with oral liquid from a basin, which can be water, and with the patient inclined and head to one side, opens patient's mouth, inserts the sponge in the basin to draw the liquid and cleans successively in the oral cavity as well as moisturizing the same.

The patient's mouth, especially critically ill patient, usually cannot moisten the oral cavity via gargling, so an implement is required. The attendant typically uses a basin, which is assumed to be clean, but bears risk of contamination from the environment which may exist in the heallth care facility, such as a COVID scenario. The facility may provide a disposable basin to fill with a liquid, but this also leaves the liquid exposed to the environment and leaves open the possibility of contamination. After the use of the implement, it is discarded and the basin is either discared or washed. While this type of implement is useful and the current standard, there remains a need to improve the art. The instant invention aims to improve the art and field.

### SUMMARY OF INVENTION

It is an object to improve treatment of oral care for a patient.

A further object is to enhance an implement for use in oral care of a patient.

Another object is to reduce the risk of cross contamination of implements used for treating oral care of a patient.

Accordingly, the instant invention is directed to a disposable oral sponge. The disposable oral sponge includes a handle which is preferably flexible and connected to a sponge head. The flexible handle includes a preloaded amount of fluid for introduction into the sponge head. The flexible handle can include a hollow conduit which is communicable with a fluid storage area of the handle. A rupturable membrane can preferably be disposed in the handle between the fluid storage area and the hollow conduit. The rupturable membrane can be ruptured by applying pressure, either on the membrane or about the rupturable membrane causing this to break and allow the flow of fluid to the sponge head. The fluid can be water, flavored water, or an oral antiseptic for example. The disposable oral sponge can preferably be contained in a sterile package prior to use.

Other objectives and features will be apparent from viewing the drawings and reading the dscription below.

### BRIEF DESCRIPTION OF THE DRAWINGS

FIG. 1 is a perspective view of the invention.

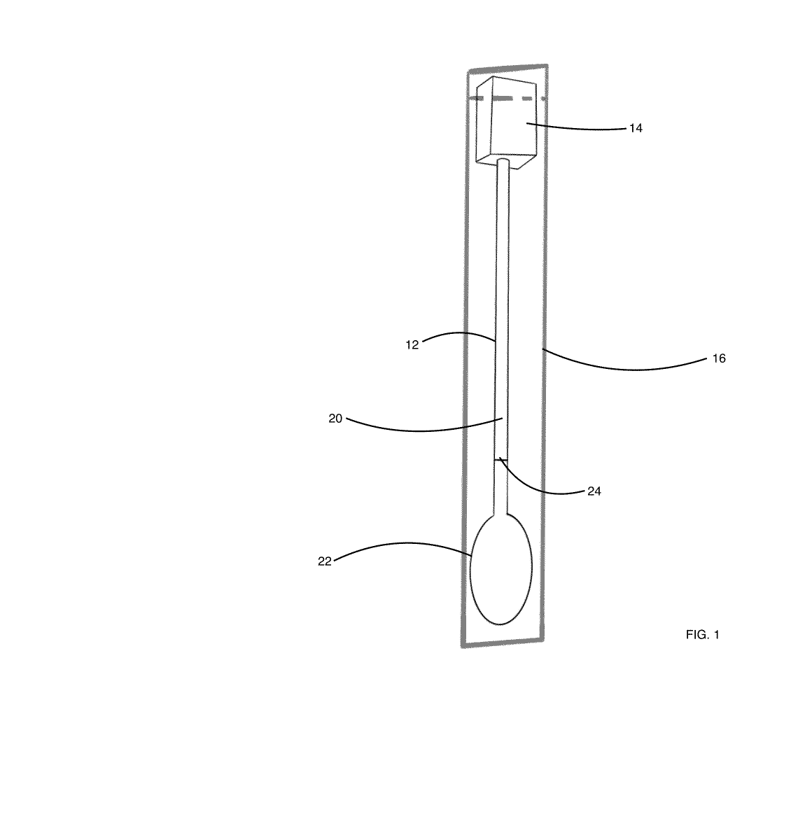

FIG. 2 is a perspective view of part of the invention in an unused mode.

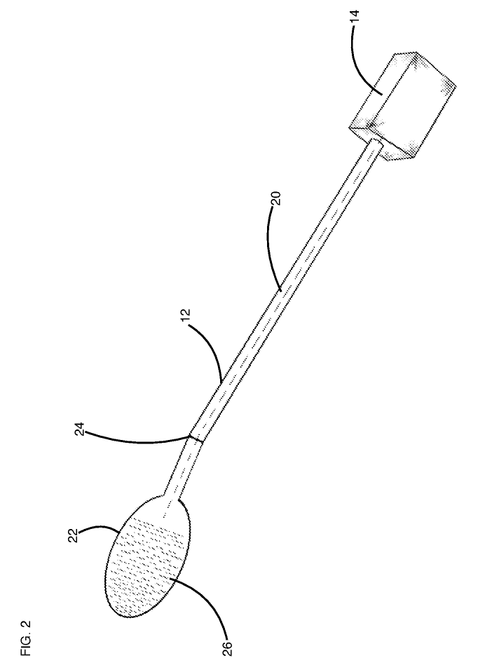

FIG. 3 is perspective view depicting a mode of releasing fluid flow in the invention.

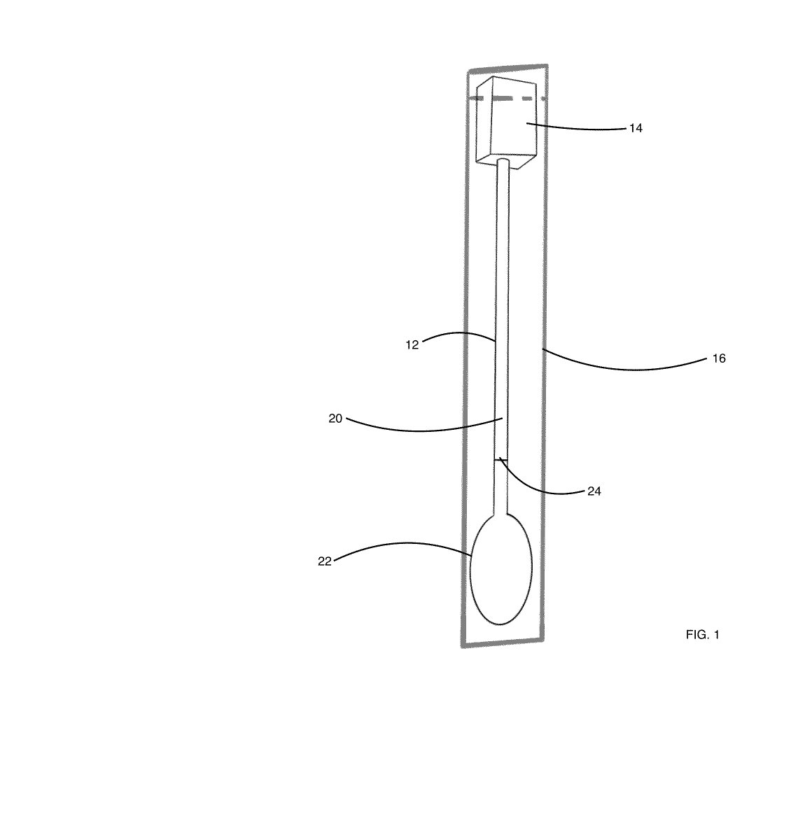

### DETAILED DESCRIPTION OF THE PREFERRED EMBODIMENT

Referring now to the drawings, a packaged disposable oral sponge is generally depicted by the numeral 10. The packaged disposable oral sponge 10 includes a flexible handle 12, connected to a sponge head 14 and is preferably contained in a sterile package 16 prior to use.

The flexible handle 12 can include a preloaded amount of fluid 18 for introduction into the sponge head 14. The flexible handle 12 can include a hollow conduit 20 which is communicable with a fluid storage area 22 of the handle 12. A rupturable membrane 24 can preferably be disposed or formed in the handle 12 between the fluid storage area 22 and hollow conduit 20 as part of a manufacturing process after loading the fluid storage area 22 with fluid 26. The rupturable membrane 24 can be ruptured by applying pressure either to the fluid storage area 22 or about the rupturable membrane 24 causing this to break and allow the flow of fluid 26 to the sponge head 14.

The fluid 26 can be water, flavored water, or an oral antiseptic for example. In the shown embodiment, the fluid storage area 22 can be a bulbous part of the handle 12. The handle 12 can be formed of a bio-friendly and sterile plastic which is flexible. The rupturable membrane 24 can be ruptured by applying pressure, for example, by squeezing the fluid storage area 22 of the handle wherein the rupturable membrane 24 is configured to be of a more fragile durometer and/or thickness of material than a remainder of handle 12 thereby imparting a weakness which enables this to rupture prior to another part of the handle 12. The handle 12 is made of a polymer material that is sufficiently flexible to allow a user to squeeze the same with manual force by hand, to compress the enclosed interior volume and apply or increase the pressure on the fluid 26 in the fluid storage area 22. For example, the handle 12 can be made of a thermoplastic material, such as a thermoplastic rubber.

The sponge head 14 may be made of any suitable material, for example, a soft, flexible, or resilient material, such as, but not limited to, closed cell foam, open cell foam, self-skinning open or closed cell foams, cast, aerated, or extruded silicone or urethane, polyurethane, or the like. The sponge head 14 may be attached to the handle 12 in any suitable manner including, but not limited to welding, adhesives, friction or press fittings, or the like. In some embodiments, the sponge head 14 is attached to the handle 12 by heat sealing or other mechanisms that do not require additional materials, to minimize potential contaminants.

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description.Thus, the previous description is not intended to be limited to the implementations shown herein but is to be accorded the widest scope consistent with the principles and novel features disclosed herein.

What is claimed is:

1. A disposable oral sponge which includes:
a flexible handle;
a sponge head connected to said flexible handle, wherein said flexible handle includes a preloaded amount of fluid for introduction into said sponge head.

2. The disposable oral sponge of claim 1, wherein said flexible handle includes a hollow conduit in communication with a fluid storage area of said handle.

3. The disposable oral sponge of claim 2, which includes a rupturable membrane disposed in said handle between said fluid storage area and said hollow conduit.

4. The disposable oral sponge of claim 3, wherein said rupturable membrane is rupturable by applying pressure to said rupturable membrane causing rupture thereof and allowing flow of fluid to the said sponge head.

5. The disposable oral sponge of claim 1, wherein said fluid is one of water, flavored water, and an oral antiseptic.

6. The disposable oral sponge of claim 1, which further includes a sterile package containing said disposable oral sponge.
